(19) United States
(12) Patent Application Publication
(10) Pub. No.: US 2009/0175842 A1
(43) Pub. Date: Jul. 9, 2009

(54) DIETARY ANTI-BACTERIAL COMPOSITIONS

(76) Inventors: Chandrakant Laxminarayan Rathi, Maharashtra (IN); Jai Shankar Arya, Maharashtra (IN); Shilpa Prasanna Risbud, Maharashtra (IN)

Correspondence Address: LADAS & PARRY LLP, 224 SOUTH MICHIGAN AVENUE, SUITE 1600, CHICAGO, IL, 60604, US

(21) Appl. No.: 12/373,096

(22) PCT Filed: Jul. 10, 2007
(86) PCT No.: PCT/IN2007/000283

§ 371 Date: Jan. 9, 2009

(30) Foreign Application Priority Data
Jul. 11, 2006 (IN) 1105/MUM/2006

(51) Int. Cl. A61K 38/48

(52) U.S. Cl. 424/94.63

(57) ABSTRACT

A synergistic dietary antibacterial composition comprising the combination of proteolytic enzymes and one or more antibiotics effective against Helicobacter pylori, useful for the prevention or treatment of gastrointestinal disorders like peptic ulcer, gastric cancers and gastritis in the intestine caused by the Helicobacter pylori is disclosed in the present invention.

### TECHNICAL FIELD OF THE INVENTION

The present invention relates to dietary antibacterial synergistic compositions comprising proteolytic enzymes and antibiotics, for the treatment and prevention of Helicobacter pylori (H. pylori) infections and associated disorders such as peptic ulcers, gastric cancer and chronic active gastritis in intestine. The present invention specifically relates to dietary compositions having synergistic effect with antibiotics effective against H. pylori infections.

### BACKGROUND AND PRIOR ART

Helicobacter pylori is a gram negative spiral rod shaped bacterium having flagella at one end and colonizing in the human gastric mucosa. B. J. Marshall and J. R. Warren in Australia reported in 1983 that this bacterium was frequently detected in stomach biopsy specimen from patients with gastritis or gastric ulcers. At that time this bacterium was named Campylobacter pylori since it resembles Campylobacter in morphology and growth characteristics. Later it was found that the bacterium is different from. Campylobacter in the fatty acid composition of its outer membrane and sequence of ribosomes 16S-RNA. Therefore, new genus of Helicobacter was established to accommodate the new found bacterium which was then referred to as H. pylori.

Strains of H. pylori are main cause of gastrointestinal diseases for instance peptic ulcers, gastric cancer and chronic active gastritis in intestine has been a major breakthrough in the gastroenterology. H. pylori strains are sensitive to various antibacterial agents intrinsically sulphonamide trimethoprim, polymixin, nalidixic acid and vancomycin, but in current treatment system two antibiotics amoxicillin and clarithromycin along with the proton inhibitors are given for a week. In other treatment bismuth salt, meteronidazole, amoxicillin, rifamycin and tetracycline are also used. However this treatment may fail for several reasons such as poor patient compliance, low gastric pH, and high bacterial load in humans and multi drug resistance (MDR) in the H. pylori strains.

To overcome these side effects, several compositions based on plant extracts having bactericidal actions on H. pylori have been developed.

CN1634320 discloses a Chinese traditional medicine composition for resisting Helicobacter pylori comprising of dandehon herb 10-20 g, astragalus root 6-8 g, and flavescent sophora root 8-12 g, clove 2-5 g.

JP2001122793 discloses a natural product derived from a stevia with bactericidal action against Helicobacter pylori, a causative bacterium of gastric cancer and gastric ulcer, without any adverse effects.

U.S. Pat. No. 5,618,564 discloses a method for the treatment of Helicobacter pylori infection wherein a composition containing protease and an antibacterial agent as active ingredients was employed to remove Helicobacter pylori from a stomach at high probability without causing side effects or the occurrence of resistant bacteria, for the treatment and prevention of peptic ulcer and recurrence of peptic ulcer caused by Helicobacter pylori infection.

Currently there are no standard sensitivity testing for these fastidious organisms; moreover interpretative criteria for the susceptibility or resistance have yet to be standardized. It is very important however, to assess antimicrobial resistance when therapy failure has occurred. Susceptibility testing is required before administering a second course of treatment.

Modern concepts in biotechnology have extended the role of enzyme to new application including bio-therapeutics. Today, enzymes are increasingly used in cosmetics for the removal of dead cells from skin and in tooth paste owing to their antibacterial and antifungal properties. The use of enzymes such as streptokinase and urokinase for dissolving clots in patients with cardiovascular diseases is well documented and is in the common practice. There is a remote probability in stimulating the evolution of drug resistance development in pathogenic microorganisms while using enzyme based treatment that operate in a manner different from antibiotics.

With this background the present invention aims at providing synergistic dietary composition and evaluating it for exhibiting inhibitory activity against H. pylori.

### DETAILED DESCRIPTION OF DRAWINGS

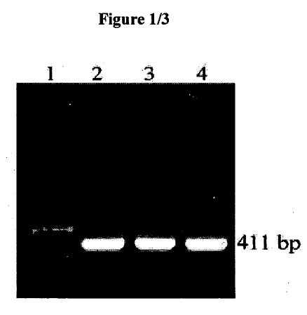

FIG. 1: PCR (ureaA gene of H. pylori) showing 411 bp amplified product. Lane 1, 100 bp DNA ladder; Lane 2 Positive control (ATCC 26695); Lane 3 and 4 genomic DNA from H. pylori clinical isolates.

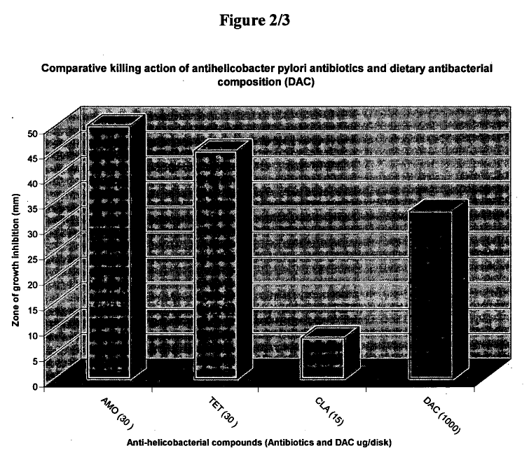

FIG. 2: Comparative zone of growth inhibition of antibiotic amoxicillin (AMO), tetracycline (TET), clarithromycin (CLA) and dietary antibacterial composition (DAC) alone against clinical isolates Helicobacter pylori 1735 by well/disc diffusion methods.

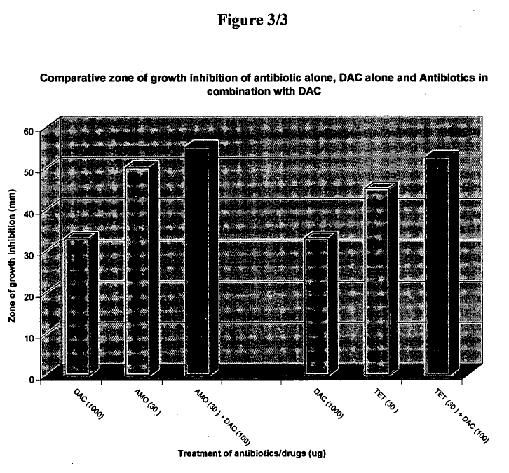

FIG. 3: Comparative zone of growth inhibition of antibiotic amoxicillin (AMO), tetracycline (TET) and dietary antibacterial composition (DAC) alone and synergistic action in combinations against clinical isolates Helicobacter pylori 1735 by well/disc diffusion methods.

### OBJECT OF THE INVENTION

The main object of the invention is to provide dietary antibacterial synergistic compositions containing proteolytic enzymes and antibiotics active against H. pylori having synergistic effect.

It is yet another object of the present invention to provide dietary anti-bacterial compositions for the treatment and prevention of H. pylori infections and associated disorders such as peptic ulcers, gastric cancer and chronic active gastritis in intestine.

Further the object of the present invention is to provide lower dosages of antibiotic during the treatment of H. pylori infection for effective synergistic action.

It is yet another object of the invention to provide molecular identification of clinical isolates of H. pylori by polymerase chain reaction (PCR).

Further the object of the present invention is to reduce the risk and rate of drug resistance as well as drug associated side effects in infections caused by the bacterium H. pylori and thus providing improvement in the health of the patient during the chemotherapy of infectious diseases.

### SUMMARY OF THE INVENTION

The present invention discloses dietary antibacterial synergistic compositions exhibiting a high degree of inhibitory activity against the bacterium H. pylori. The present invention provides synergistic compositions comprising proteolytic enzymes and antibiotics. The present invention also provides an antibacterial composition active against H. pylori suitable for the treatment and prevention of pathological conditions associated with H. pylori. The compositions, typically provides treatment against infections and H. pylori related gastrointestinal disorders such as peptic ulcers, gastric cancer and chronic active gastritis in intestine.

Furthermore the said composition reduces multiple drug resistance (MDR) as well as drug associated side effects and toxicity in humans and animal thereby providing dietary antibacterial supplements for health benefits.

### DETAILED DESCRIPTION OF THE INVENTION

As used herein, the following term is to be taken to have the following meaning:

Dietary Antibacterial Composition (DAC)—refers to enzyme formulations having serratiopeptidase and fungal protease.

Dietary Antibacterial Synergistic compositions—refers to compositions comprising Dietary Antibacterial Composition (DAC) and at least one antibiotic.

The present invention describes compositions for the treatment and prevention of pathological conditions associated with H. pylori. The present invention describes synergistic dietary compositions active against bacteria H. pylori and their use in the treatment of gastrointestinal disorders such as peptic ulcers, gastric cancer and chronic active gastritis in intestine.

The dietary antibacterial synergistic composition comprising of proteolytic enzymes along with antibiotics act as antibacterial for the treatment of H. pylori related infections having synergistic action with antibiotic exhibiting anti H. pylori activity.

The compositions of the present invention comprise of one or more enzymes selected from serratiopeptidase and fungal protease like. The fungal protease is an acid protease.

According to the present invention, the antibiotics employed are selected from the group of drugs exhibiting anti H. pylori activity such as amoxicillin, tetracycline, clarithromycin alone or in combination thereof.

The said dietary antibacterial compositions exhibit synergistic effect when administered along with antibiotics and effective against the bacterium H. pylori even if lower dosages of antibiotic are administered during the treatment of H. pylori infection. The said compositions therefore reduce multiple drug resistance (MDR) and drug associated side effects thereby providing health benefits.

The present invention further deals with molecular identification of clinical isolates of H. pylori by polymerase chain reaction (PCR) and antibacterial activity of individual component comprising the dietary antibacterial composition. The present invention also deals with sensitivity of standard antibiotics for the treatment of H. pylori related disorders against H. pylori strains viz, wild type and clinical isolates.

In one aspect, the present invention describes the effect of poly enzyme formulations on drug susceptibility of H. pylori and the efficacy of antibiotics. These formulations contain various proteolytic enzymes like acid protease and serratiopeptidase derived from natural origin like fungi and bacteria. These proteolytic enzymes are effective at specific range of pH.

In another aspect, the present invention describes the sensitivity profiles of dietary antibacterial composition against H. pylori along with the clinical isolates performed by the disc diffusion assays to study antibacterial effect against H. pylori strains. Also the studies for synergistic action of the said composition when used along with various antibiotics that are effective against H. pylori are described in the present invention.

Enzymes: A live cell is the unit of our body. Many biochemical reactions take place in a single cell. Every reaction is catalyzed by the special kind of protein molecules known as enzymes, are very efficient catalysts for biochemical reactions. Enzymes speed up these reactions by providing an alternative reaction pathway of lower activation energy.

Like catalysts, enzymes also participate in the reaction and provide an alternative reaction pathway without undergoing any permanent changes till the end of the reaction but they alter the rate of reaction without altering the reaction equilibrium.

Most chemical catalysts catalyze a wide range of reactions but they are not substrate selective. In contrast enzymes are highly selective, catalyzing only specific reactions. This specificity is due to the shapes of the enzyme molecules.

Many enzymes consist of a protein and a non-protein (called the cofactor). The proteins in enzymes are usually globular. The intra and intermolecular bonds that hold proteins in their secondary and tertiary structures are disrupted by changes in temperature and pH which affects shape of the enzyme protein and therefore the catalytic activity of an enzyme is pH and temperature sensitive.

Proteases: Proteases (proteinases) are large group of enzymes that break or hydrolyse the peptide bonds in the polypeptide chain of various protein molecules. These enzymes are involved in a multitude of physiological reactions from simple digestion of food proteins to highly regulated cascades. Peptidases can break either specific peptide bonds (limited proteolysis), depending on the amino acid sequence of a protein, or break down a complete peptide to amino acids (unlimited proteolysis). The activity can be a destructive change abolishing a protein's function or digesting it to its principal components. It can be an activation of a function or it can be a signal in a signalling pathway. Proteases are also a type of exotoxin, which is a virulence factor in bacteria pathogenesis. Bacteria exotoxic proteases destroy extracellular structures.

In present days, applications of enzymes are well known and used for nutritional purpose and in many diseases. These include: Reducing inflammation, Cleansing the blood of debris, Dissolving fibrin in the blood, reducing the risk of clots, maximized immune system, killing of bacteria, viruses, molds and fungi, elimination of autoimmune diseases, dissolving of scar tissue.

Some times our body is deficient to secrete the key/basic enzymes resulting significant change occurs in enzymes balance ultimately causes disorders or diseases. Patients take medicine to fight with the disorders/diseases but antibiotics/drugs don't work properly due to improper secretions of enzymes/hormones.

Based on enzyme studies and their significant role in life processes, inventors have attempted at eliminating antibiotic/drugs associated problems like higher doses of antibiotic for treatment, unwanted antibiotic/drug load in body and multiple drug resistance (MDR) in infectious agents by developing a novel synergistic formulation, based on proteolytic enzymes along with reduced dose of antibiotic to provide proper requirement of enzymes and antibacterial component required by the body effective to fight with infectious agents.

Accordingly, the present invention describes dietary antibacterial synergistic composition comprising proteolytic enzymes and anti-bacterial agents (antibiotics/drugs) derived from natural, semi-synthetic or synthetic sources acts against gram negative bacteria H. pylori for the treatment and prevention of pathological conditions associated with H. pylori. The said synergistic compositions are also active against bacteria H. pylori and their use in the treatment of gastrointestinal disorders such as peptic ulcers, gastric cancer and chronic active gastritis in intestine.

The present investigation is more specifically explained by following examples. However, it should be understood that the scope of the present invention is not limited by the examples in any manner. It will be appreciated by any person skilled in this art that the present investigation includes the following examples and further can be modified and altered within the technical concept of the present investigation.

### EXAMPLES

### Example 1

### Molecular Identification of H. pylori by Polymerase Chain Reaction (PCR)

Two strains of Helicobacter pylori (HP-1734, HP-1735) were isolated from the patients with duodenal ulcer, attending the endoscopy units in the gastroenterology Departments, SGPGIMS for the upper gastrointestinal complaints, were used in the study. None of the patients had received any antimicrobial therapy for at least four weeks prior to the study.

a) Culture of H. pylori from Gastric Biopsy Specimens:

For Helicobacter pylori culture, gastric biopsy samples were homogenized and cultured on Brucella chocolate agar (Difco) containing antibiotic supplement (vancomycin 6 mg/L, polymixin-B 2500 IU/L, and amphotericin B 2 mg/L) as described earlier with 7% sheep blood (13-16) incubated at 37° C. for 5 to 10 days under the micro-areophilic condition (5% O₂, 10% CO₂, and 85% N₂). Organism was identified as H. pylori based on colony morphology (small, trasluscent colonies) gram's staining (Gram −ve,), and positive rapid urease test oxidase and catalase (14-16). H. pylori ATCC 26695 was used as a control strain.

### (b) DNA Extraction:

DNA extraction was carried out as per the methods of Sambroke et al. (Molecular cloning: A laboratory Manual, volume 3, Cold spring Harbor Laboratory Press, N Y, 2^{nd} Ed. 1989: 18.1-18.86. 1989). Briefly, bacterial pellet was suspended in 100 μl ice colds solution I (Glucose 50 mM, Tris HCL 25 mM and EDTA 10 mM) and after mixing by overtaxing 200 μl freshly prepared solution II (NaOH 2 N and SDS 10%) was added to each tube and gently mixed by inverting the tube 10-15 times. Tubes were centrifuged at 12,000 rpm for 10 min at 4° C. and supernatant collected in a fresh tube DNA was purified by equal volume of Phenol:Chloroform:Isoamyl alcohol (25:24:1) and precipitated with two volumes of absolute ethanol. The precipitated DNA was re-suspended in 70% ethanol and centrifuged at 12,000 rpm for 10 min at 4° C. and pellet DNA was dissolved in 50 μl of distilled water and stored at −20° C. till use.

### (c) Polymerase Chain Reaction (PCR)

ureA gene PCR (product size 411 bp) was performed for molecular identification of H. pylori with the following primer sequence (17).

Forward: 5′-GCCAATGGTAAATTAGITT-3′
Reverse: 5′-CTCC TTAATTGTTTTTAC-3′

Amplification was performed in 50 μl reaction mixture volume containing 20 pM concentration of each oligonucleotide primer, 200 μM of each dATP, dCTP, dGTP and dTTP, 10× reaction buffer, 10° C. of template DNA (100 ng DNA) and 2.5 units of Tag DNA polymerase enzyme. PCR conditions included 35 cycles each of de-naturation at 94° C. for 1 min annealing at 45° C. for 1 min and extension at 72° C. for 1 min, followed by final extension at 72° C. for 5 min. 10 μl of the PCR product was analyzed by electrophoresis on 1.5% agarose gels stained with ethidium bromide 0.5 μg/ml and visualized under UV illumination. Electrophoresis was performed in TAE buffer.

Both the clinical isolates were confirmed to be H. pylori by ureA PCR as shown in FIG. 1.

### d) Urease Test:

Urease activity of the H. pylori wild type and the clinical isolates were determined quantitatively using the rapid urease test (RUT) by suspending bacteria in a mixture (1:3) of 0.02% (w/v) cresol red 0.1% (w/v) EDTA and 1.5% (w/v) urea. With urease positive bacteria, a colour change of the reaction solution from yellow to purple was observed within a minute. Two clinical isolates HP-1734 and HP-1735 were identified as H. pylori on the basis of morphological and biochemical examination. Both strains showed the urease and catalase activities.

### Example 2

### Antibiotic Susceptibility Test by Disc Diffusion Methods

Antimicrobial sensitivity of isolated H. pylori and the reference strain (ATCC 26695) was detected by conventional agar disc diffusion methods (9, 10 and 13). The bacterial suspension (McFarland tube No. 3) of H. pylori was plated on Brucella chocolate agar plates. The Discs (6 mm diameter, Hi-Media India) of different antibiotics (amoxicillin 30 μg/disc, tetracycline—30 μg/disc and clarithromycin—15 μg/disc) were placed on the plates and incubated at 37° C. in microaerofilic condition for 72 hrs and examined for the diameter of the inhibition zone, which was measured in millimeters with the measuring caliper and noted. The methods based on the NCCLS guidelines for the fastidious organism H. influenza. Zone size ≦18 mm was considered resistant for amoxicillin (an analog of ampicillin); ≦30 mm for clarithromycin and tetracycline (16, 18-20). For the purpose of analysis, the non susceptible categories of intermediate and resistant were combined into a single resistance category.

H. pylori strain HP-1734 was found to be sensitive to all the four drugs tested. Strain HP-1735 was sensitive to amoxicillin, ciprofloxacin, and tetracycline and resistant to clarithromycin (Table 1). Further studies were conducted with strains HP-1735.

### Example 3

### Polyenzyme Formulation

Three different polyenzyme formulations developed by Advanced Enzyme Technologies Ltd. Thane, were tested to see their effect on growth of H. pylori. These formulations contain various proteolytic enzymes e.g., Acid Protease and Serratiopeptidase derived from natural origin like fungi and bacteria and work at specific range of pH.

### Specifications of Active Formulation:

Description: Off white amorphous powder having characteristic odour. Solubility: Soluble in water 0.9%. NaCl or phosphate buffer saline

### Pathogens: Absent

### Stock: 100-500 mg

Formulation was tested at three different concentration (1, 5 and 10 mg/ml) in broth culture and on brucella chocolate agar medium to observe their effect on growth of H. pylori. The formulation were dissolved in phosphate buffered saline and added to the medium after cooling it to 56° C. to avoid inactivation of the enzymes. The experiments were repeated three times to confirm the results.

### Example 4

### Effect of Poly Enzyme Formulations on Drugs Susceptibility of H. pylori

Both the strains were tested with 100, 500, 1000 μg/ml concentration of the three formulation to study the inhibitory effect. It was observed that out of three, only one formulation with 1000 μg/ml concentration had significant inhibitory effect. Therefore, further experiments were carried out with 1000 μg/ml concentration of dietary antibacterial composition (DAC).

### Example 5

### Dietary Antibacterial Composition (DAC)

A dietary antibacterial composition contains proteolytic enzymes consisting of acid protease and serratiopeptidase at various concentrations.

Dietary Antibacterial synergistic composition: Dietary Antibacterial Synergistic composition for the prevention of gastrointestinal disorder induced by the H. pylori comprises of DAC and at least one or more antibiotic in combination selected from a group such as amoxicillin, tetracycline and clarithromycin preferably amoxicillin and tetracycline.

### Enzyme Used at Concentration Level:

Acid stable protease in the range of about 1-3 mg
Serratiopeptidase in the range of about 0.5-2 mg
Antibiotic used at minimal inhibitory concentration level:
Amoxicillin (30 μg/disc)—Ready made disc was used. Tetracycline (30 μg/disc)—Ready made disc was used. Clarithromycin (15 μg/disc)—Ready made disc was used.

### Example 6

### Evaluation of Dietary Antibacterial Composition

a) Evaluation of Antibacterial Activity of the Dietary Antibacterial Composition Against H. pylori.

Dietary antibacterial composition showed the antibacterial activity against H. pylori (Wild Type—ATCC-26695) and two clinical isolates (HP-1734 and HP-1735) by disc diffusion assays (in-vitro). The effective concentration of antibacterial dietary composition used is 100-1000 mg/ml and it showed the activity in terms of zone of growth inhibition in Table 1.

TABLE 1
Antibacterial sensitivity profile of dietary antibacterial
composition against H. pylori wild type and
clinical isolates by the well/disc diffusion method.

| | | Growth condition | | |
|---|---|---|---|---|
| | | at various | | |
| | Bacterial strains | concentration of | | |
| S. No. | (Helicobacter pylori) | 100 | 500 | 1000 |
| 1. | Wild Type - ATCC-26695 | + | ± | − |
| 2. | Clinical isolates - 1734 | + | ± | − |
| 3. | Clinical isolates - 1735 | ± | − | − |

+ = Resistance,
− = Sensitive,
f = Inhibitory

Clarithromycin showed less zone of growth inhibition as compared the dietary antibacterial composition (Table 2). DAC at least showed 4.1 fold more killing activity against H. pylori clinical isolates 1735. DAC showed comparatively less killing activity then amoxicillin and tetracycline against H. pylori clinical isolate 1735 (FIG. 2).

TABLE 2
Showing zone of growth inhibition (mm) produced by dietary
antibacterial composition against H. pylori (clinical isolate
1735) by the well/disc diffusion method.

| | Antibiotic concentration | | | |
|---|---|---|---|---|
| | (gg/disk) | | | DAC |
| Bacterial strains | | | CLA | concentration @ |
| (Helicobacter pylori) | AMO (30) | TET (30) | (15) | 1000 μg/well |
| Clinical isolates - 1735 | 50 | 45 | 8 | 33 |

AMO = Amoxicillin;
TET = Tetracycline;
CLA = Clarithromycin;
DAC = Dietary Antibacterial Composition

DAC exhibits synergistic effect with amoxicillin and tetracycline showing 10% and 16.5% more growth inhibition against Helicobacter pylori 1735 respectively. In comparison to amoxicillin, dietary antibacterial composition showed 6.6% more growth inhibition when administered with tetracycline table 3. Further clarithromycin when used along with dietary antibacterial composition failed to show any synergistic effect against clinical isolates H. pylori 1735. The comparative zone of growth inhibition for amoxicillin, tetracycline and dietary antibacterial composition (DAC) when taken alone and their growth inhibition representing synergistic action when used in combination was plotted into a graph as shown in FIG. 3.

TABLE 3
Antibacterial activity of dietary antibacterial composition (DAC)
along with standard antibiotics against clinically isolated strain
Helicobacter pylori 1735 by well/disc diffusion methods.

| | | Zone of growth of | |
|---|---|---|---|
| | Dietary antibacterial | inhibition (mm) | |
| | composition @ 100 μg/well | | Percentage (%) |
| S. No | along with antibiotics | Net zone | enhancements |
| 1. | AMO (30) + DAC (100) | 55.0 | 10.0 |
| 2. | TET (30) + DAC (100) | 52.5 | 16.6 |

AMO = Amoxicillin;
TET = Tetracycline;
DAC = Dietary Antibacterial Composition

### b) Evaluation of Proteolytic Activity of Dietary Antibacterial Compositions:

Note: One bacterial protease unit (PCU) is defined as that quantity of enzyme that produces the equivalent of 1.5 μg per ml of L-tyrosine per min under the conditions of the assay and the protease activity is expressed in PC units.

Stock solutions containing 100.0, 75.0, 50.0 and 25.0 μg of tyrosine per ml in 0.006N of hydrochloric acid were prepared. 10 ml casein substrate (0.7%) was treated with 2 ml of enzyme solution containing 10-44 PC unit prepared in tris buffer pH-7.0 at 37° C. for 30-min. The reaction was terminated by using 10 ml TCA solution. Un-hydrolyzed casein was removed by filtration using whatman filter paper no. 42. The solubilized casein was determined spectrophotometrically by recording the absorbance of all the four solutions at 275 nm in 1-cm cell on a suitable spectrophotometer versus 0.006N hydrochloric acid. The value of absorbance versus tyrosine concentration were plotted to obtain standard curve of tyrosine.

From the Standard Curve, the absorbance of a solution having a tyrosine concentration of 60 micro g per ml was determined by interpolation. A value close to 0.0115 was obtained. The interpolated value was divided by 40 to obtain the absorbance equivalent to that of a solution having a tyrosine concentration of 1.5μ/ml, and the value thus derived was recorded as AS.

PC/g=(AU/AS)×(22/30W)

### Where:

22=final volume, in ml, of the reaction mixture;
30=the time of the reaction, in minutes
W=the weight of the sample g/2 ml

A_{U}=Absorbance of unknown sample(Absorbance of test sample−Absorbance of blank sample)

Dietary antibacterial composition comprising various concentrations of serratiopeptidase and fungal proteases showing ranges in proteolytic activity are shown in table 4.

TABLE 4

| | Total activity | Serratiopeptidase | Fungal protease | |
|---|---|---|---|---|
| S. No. | (PCU/g) | (PCU/g) | (PCU/g) | Lactose (mg) |
| 1. | 5,00,000 | 2,00,000 | 3,00,000 | Q.S. |
| 2. | 4,00,000* | 2,50,000 | 1,00,000 | Q.S. |
| 3. | 75,000 | 50,000 | 7,000 | Q.S. |

*= Shows optimum antibacterial activity against Helicobacter pylori.
Q.S. = Quantity sufficient

### Example 5

### Stability Studies of Dietary Antibacterial Compositions

Stability studies of active dietary antibacterial compositions was done up to 90 days at temperature 25° C.±2% & relative humidity 60% f 5.0% for 90 days as shown in table 5.

TABLE 5
Stability studies of active dietary antibacterial composition was
done up to 90 days at temperature 25° c. + 2% &
relative humidity 60% ± 5.0%.

Activity (PC/g) on alternative days

| Formulations | 0 | 30 | 60 | 90 |
|---|---|---|---|---|
| Dietary antibacterial | 258233 | 257982 | 257834 | 257748 |
| Compositions | | | | |

1. A synergistic dietary antibacterial composition for the treatment or prevention of gastrointestinal disorders like peptic ulcer and gastric cancers and gastritis in the intestine caused by Helicobacter pylori, comprising proteolytic enzymes in combination with at least one antibiotic, wherein the proteolytic enzymes are serratiopeptidase and fungal protease.

2. The composition as claimed in claim 1, wherein the proteolytic activity of serratiopeptidase and fungal protease against gastrointestinal disorders caused by the Helicobacter pylori ranges from 100000 to 500000 PCU/g, preferably 200000 to 300000 PCU/g.

3. The composition as claimed in claim 2, wherein the serratiopeptidase is originated from Serratia marcescens.

4. The composition as claimed in claim 2, wherein the proteolytic activity of serratiopeptidase ranges from 10,000 to 2,50,000 PCU/g.

5. The composition as claimed in claim 2, wherein the fungal protease is an acid protease.

6. The composition as claimed in claim 5, wherein the fungal protease is originated from Aspergillus oryzae, Aspergillus niger.

7. The composition as claimed in claim 2, wherein the proteolytic activity of fungal protease ranges from 5000 to 400000 PCU/g.

8. The composition as claimed in claim 1, wherein the antibiotic is selected from the group consisting of amoxicillin, tetracycline, clarithromycin or any other drug effective against Helicobacter pylori, alone or in combinations thereof.

9. The composition as claimed in claim 1, wherein the said composition is further effective against clinically isolated Helicobacter pylori.

10. The composition as claimed in claim 1, wherein the said composition comprises 10 to 1000 μg/ml of the antibiotic active formulation.

11. The composition as claimed in claim 1, wherein the preferable dosage of natural dietary antibacterial composition ranges from 500 to 1000 μg/ml.
